(19) United States
(12) Patent Application Publication
(10) Pub. No.: US 2024/0369583 A1
(43) Pub. Date: Nov. 7, 2024

(54) ALZHEIMER'S DISEASE BIOMARKER, AND SCREENING METHOD THEREFOR AND APPLICATION THEREOF

(71) Applicant: SHENZHEN INSTITUTES OF ADVANCED TECHNOLOGY CHINESE ACADEMY OF SCIENCES, Shenzhen (CN)

(72) Inventors: Yu CHEN, Shenzhen (CN); Yijing CHEN, Shenzhen (CN); Yingying FAN, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 18/689,508

(22) PCT Filed: Sep. 16, 2021
(86) PCT No.: PCT/CN2021/118739

§ 371 Date: Mar. 6, 2024

(30) Foreign Application Priority Data
Sep. 7, 2021 (CN) 202111042127.6

(51) Int. Cl. G01N 33/92; G01N 30/02; G01N 30/72; G01N 30/88

(52) U.S. Cl. CPC G01N 33/92; G01N 30/7233; G01N 30/88; G01N 2030/027; G01N 2030/8813; G01N 2800/2821

(57) ABSTRACT

An Alzheimer's disease biomarker, and a screening method therefor and use thereof. The Alzheimer's disease biomarker is 11(Z), 14(Z)-eicosadienoic acid. The screening method for the Alzheimer's disease biomarker comprises: acquiring samples, testing the samples, and performing structural identification and data analysis on metabolites in the samples, wherein specifically, metabolites in faeces samples can be tested by liquid chromatography-mass spectrometry, to carry out structural identification on the metabolites; and selecting a metabolite showing a difference from the metabolite level of a control group as the Alzheimer's disease biomarker. Using 11(Z), 14(Z)-eicosadienoic acid as an Alzheimer's disease biomarker is of great significance to clinical early diagnosis of Alzheimer's disease; the present invention can be used for preparing a diagnostic tool for Alzheimer's disease, predicting the individual treatment effects of patients with Alzheimer's disease, and preparing drugs for treating Alzheimer's disease.

### TECHNICAL FIELD

The present application belongs to the technical field of biomarkers and, specifically, relates to an Alzheimer's disease biomarker, a screening method therefor and use thereof.

### BACKGROUND

Recent studies have found that multiple neuropsychiatric diseases such as Parkinson's disease, depression and autism are related to an imbalance in intestinal flora and more than 80% of patients with Alzheimer's disease (AD) have an imbalance in intestinal flora, indicating that a homeostasis of the intestinal flora is closely related to pathogenesis of neurodegenerative diseases such as Alzheimer's disease.

CN111197085A discloses a group of autism-related intestinal flora biomarkers and use thereof. It is found that intestinal flora can assist in degradation of intestinal toxins, thereby reducing damage of mitochondria caused by the accumulation of the toxins and occurrence of autism induced by the damage. Metagenome sequencing analysis was performed on a stool sample of a patient. It was found that significant differences in abundance were between metabolic pathways including degradation of multiple toxins such as methylphosphonate, 3-phenyl propionate, 3-(3-hydroxyphenyl)methyl propionate, methylglyoxal, halohydrocarbon, p-aminobenzoic acid, benzamide, styrene, naphthalene, xylene and benzoic acid and metabolic enzymes related to the metabolic pathways, and significant differences in abundance were also between metabolic pathways including synthesis of glutathione and L-glutamine and metabolic enzymes related to the metabolic pathways. A combination of biomarkers related to the occurrence of autism was obtained through screening, and the combination of biomarkers was used for constructing a random forest classification model for diagnosing autism, which has a relatively good effect on auxiliary diagnosis of autism. The results indicate that a homeostasis of the intestinal flora is closely related to pathogenesis of neuropsychiatric diseases but do not disclose a relationship between intestinal flora organisms and pathogenesis of neurodegenerative diseases such as Alzheimer's disease.

CN110333310A discloses a group of biomarkers for diagnosing Alzheimer's disease in a subject or determining a risk of occurrence of Alzheimer's disease in the subject. The biomarkers include cholic acid, chenodeoxycholic acid, allocholic acid, indole-3-lactic acid and tryptophan. Contents of the biomarkers in plasma are detected so that an early stage of Alzheimer's disease can be evaluated with high accuracy at a fast detection speed.

CN106062563A discloses a group of biomarkers for early diagnosis of Alzheimer's disease. The biomarkers include a brain-derived nerve growth factor, an insulin-like growth factor 1, a tumor growth factor β1, a vascular endothelial growth factor, an interleukin-18 and a monocyte chemoattractant protein 1. At least four particular biomarkers are detected, thereby determining levels of the biomarkers in an individual biological sample. Compared with reference levels of the biomarkers, it is determined that the levels of the biomarkers are increased or decreased, thereby using the biomarkers for indicating Alzheimer's disease.

At present, most biomarkers of the studied Alzheimer's disease are selected from peripheral blood. When detection is performed, blood of a subject needs to be collected first, and a post-processing process of the blood is complex. Therefore, screening a biomarker for early diagnosis of Alzheimer's disease and developing a convenient, quick, safe and non-invasive auxiliary diagnosis method by studying a relationship between a metabolic homeostasis of intestinal flora and pathogenesis of Alzheimer's disease have an important effect on improving accuracy of diagnosing Alzheimer's disease, an early warning of the disease, pathological typing and prediction and evaluation of a development stage.

### SUMMARY

The present application provides an Alzheimer's disease biomarker, a screening method therefor and use thereof. In the present application, the Alzheimer's disease biomarker is screened out from a fecal metabolite of an organism suffering from Alzheimer's disease. 11(Z), 14(Z)-Eicosadienoic acid is screened out as the Alzheimer's disease biomarker for auxiliary determination of a symptom of Alzheimer's disease and characterized by high detection accuracy, convenience, quickness, safety and non-invasiveness, which is of great clinical guiding significance for auxiliary diagnosis of a relevant index of Alzheimer's disease.

In a first aspect, the present application provides an Alzheimer's disease biomarker. The Alzheimer's disease biomarker is 11(Z), 14(Z)-eicosadienoic acid.

In a second aspect, the present application provides an early diagnosis model for Alzheimer's disease. An input variable of the early diagnosis model for Alzheimer's disease includes a peak intensity value of a mass spectrum of the Alzheimer's disease biomarker 11(Z), 14(Z)-eicosadienoic acid described in the first aspect.

Preferably, an output variable of the early diagnosis model for Alzheimer's disease includes a ratio of a peak intensity value of a mass spectrum of 11(Z), 14(Z)-eicosadienoic acid in a stool sample of a to-be-detected individual to a peak intensity value of a mass spectrum of 11(Z), 14(Z)-eicosadienoic acid in a stool sample of a normal individual, where when the ratio is less than 1, it is determined that the to-be-detected individual suffers from or is at risk of suffering from Alzheimer's disease.

The stool sample of the normal individual in the present application is a stool sample of a healthy individual.

The inventors unexpectedly found that a level of 11(Z), 14(Z)-eicosadienoic acid in feces of AD model mice was significantly lower than that of wild-type control mice while a level of 11(Z), 14(Z)-eicosadienoic acid in hippocampi of the AD mice was significantly higher than that of the control mice, suggesting that the change in the level of the fecal metabolites may reflect abnormalities of related metabolic pathways in brains of the AD mice and indicating that 11(Z), 14(Z)-eicosadienoic acid can be used as the Alzheimer's disease biomarker, which is of great significance for early clinical diagnosis of Alzheimer's disease.

In a third aspect, the present application provides a method for screening the Alzheimer's disease biomarker described in the first aspect. The method for screening the Alzheimer's disease biomarker includes: collecting a sample, detecting the sample, and performing structural identification and data analysis on a metabolite in the sample.

As a preferred technical solution of the present application, in the step of collecting the sample, the sample includes feces.

In the step of detecting the sample, the detection is performed through a liquid chromatography-mass spectrometry method.

Preferably, in the step of performing the structural identification on the metabolite in the sample, the structural identification is performed on the metabolite in the sample by matching with a retention time, a molecular mass, a secondary fragmentation spectrum and crash energy of a metabolite in a database, and an obtained identification result is checked and confirmed.

Preferably, in the step of performing the data analysis on the metabolite in the sample, the data analysis is performed on the metabolite in the sample through any one or a combination of at least two of univariate statistical analysis, multidimensional statistical analysis, differential metabolite screening, differential metabolite correlation analysis or KEGG pathway analysis.

Preferably, the multidimensional statistical analysis includes: constructing an orthogonal partial least squares-discriminant analysis model, and screening out a metabolite with VIP>1 from a detection result as the Alzheimer's disease biomarker.

In a fourth aspect, the present application provides a use of the Alzheimer's disease biomarker described in the first aspect to preparation of a diagnostic tool for Alzheimer's disease. The diagnostic tool for Alzheimer's disease includes an apparatus for predicting a risk of suffering from Alzheimer's disease and/or a diagnostic kit for Alzheimer's disease.

In a fifth aspect, the present application provides the apparatus for predicting a risk of suffering from Alzheimer's disease described in the fourth aspect. The apparatus includes:
a detection unit, which is configured to perform qualitative and quantitative analysis on the Alzheimer's disease biomarker described in the first aspect in a metabolite sample;
a control unit, which is configured to perform second check, confirmation and experimental data quality evaluation on a result of the qualitative and quantitative analysis of the Alzheimer's disease biomarker; and
an evaluation unit, which uses a quantitative result of the Alzheimer's disease biomarker as an input variable and provides a risk score after comprehensive calculation to predict a risk of suffering from Alzheimer's disease.

Preferably, the detection unit performs the qualitative and quantitative analysis on the Alzheimer's disease biomarker through a liquid chromatography-mass spectrometry method.

In a sixth aspect, the present application provides the diagnostic kit for Alzheimer's disease described in the fourth aspect. The diagnostic kit for Alzheimer's disease includes a reagent for measuring a content level of the Alzheimer's disease biomarker described in the first aspect.

In a seventh aspect, the present application provides a use of the Alzheimer's disease biomarker described in the first aspect to preparation of a drug for treating or diagnosing Alzheimer's disease.

It is to be noted that scientific and technical terms and abbreviations thereof used in the present application have meanings commonly understood by those skilled in the art. Some terms and abbreviations used in the present application are listed below.
KEGG: Kyoto Encyclopedia of Genes and Genomes.
VIP: Variable Importance for the Projection.
P-value: the p-value represents a p-value of significance analysis, and the smaller the p-value, the more significant the difference.
QC: Quality Control.
ESI: Electron Spray Ionization.
TOF MS: Time of Flight Mass Spectrometer.

Compared with the existing art, the present application has the beneficial effects described below.

(1) In the present application, the structural identification is performed on the metabolite in the stool sample of the organism through the liquid chromatography-mass spectrometry method, a differential lipid molecule with a biological significance is explored as the Alzheimer's disease biomarker, and the Alzheimer's disease biomarker 11(Z), 14(Z)-eicosadienoic acid is screened out. The content level of 11(Z), 14(Z)-eicosadienoic acid in the feces of the AD model mice is significantly lower than that of the wild-type control mice while the content level of 11(Z), 14(Z)-eicosadienoic acid in the hippocampi of the AD model mice is significantly higher than that of the wild-type control mice. Previous studies have not reported abnormal expression of 11(Z), 14(Z)-eicosadienoic acid in Alzheimer's disease. Using 11(Z), 14(Z)-eicosadienoic acid as the Alzheimer's disease biomarker is of great significance for the early clinical diagnosis of Alzheimer's disease.

(2) In the present application, the content level of the Alzheimer's disease biomarker is used as a detection index and used in the early diagnosis model for Alzheimer's disease for the auxiliary determination of the symptom of Alzheimer's disease. The content level of 11(Z), 14(Z)-eicosadienoic acid in the stool sample of the to-be-detected organism is detected, and it is determined that whether the to-be-detected sample suffers from or is at risk of suffering from Alzheimer's disease according to a ratio of the content level of 11(Z), 14(Z)-eicosadienoic acid in the to-be-detected sample to the content level of 11(Z), 14(Z)-eicosadienoic acid in the normal sample. The feces are a detection source obtained in a non-invasive manner, which is superior to detection means such as blood draw. This method is characterized by high detection accuracy, convenience, quickness, safety and non-invasiveness, which is of great clinical guiding significance for the auxiliary diagnosis of the relevant index of Alzheimer's disease.

### BRIEF DESCRIPTION OF DRAWINGS

FIG. 1 is a diagram illustrating peak intensities of mass spectrums of 11(Z), 14(Z)-eicosadienoic acid in fecal metabolites of AD model mice and wild-type control mice. In the figure, FWT is the stool sample of the wild-type control mice, and FTG is the stool sample of the AD model mice.

FIG. 2 is a diagram illustrating peak intensities of mass spectrums of 11(Z), 14(Z)-eicosadienoic acid in hippocampi of brains of AD model mice and wild-type control mice. In the figure, HWT is the hippocampus sample of the wild-type control mice, and HTG is the hippocampus sample of the AD model mice.

### DETAILED DESCRIPTION

Technical solutions of the present application are further described below through embodiments in conjunction with drawings. However, the following examples are only simple examples of the present application and do not represent or limit the protection scope of the present application. The protection scope of the present application is subject to the claims.

In the following examples, unless otherwise specified, the reagents and consumables used are purchased from conventional reagent manufacturers in the art; unless otherwise specified, the experimental methods and technical means used are conventional methods and means in the art.

### Example 1

This example provides a method for screening an Alzheimer's disease biomarker in feces.

### (1) Sample Processing

Stool samples were collected from 9-month-old AD model mice (APP/PS1) and wild-type control mice, and the samples were pretreated. After the collection, the stool samples were placed on dry ice for quick freezing and placed in a refrigerator at −80° C. for storage. After the samples were slowly thawed in an environment at 4° C., the to-be-detected samples were taken, a pre-cooled methanol/acetonitrile/aqueous solution (2:2:1, v/v) was added, vortexed to be mixed, sonicated for 30 min at a low temperature, left for 10 min at −20° C. and centrifuged at 14000 g for 20 min at 4° C., and supernatant was taken for vacuum drying. When mass spectrometry analysis was performed, 100 μL acetonitrile aqueous solution (acetonitrile:water=1:1, v/v) was added for reconstitution, vortexed and centrifuged at 14000 g for 15 min at 4° C., and supernatant was taken for the injection analysis.

### (2) Ultra-High Performance Liquid Chromatography-Mass Spectrometry Analysis

### Conditions for Chromatography:

The samples were separated by using a HILIC column of an Agilent 1290 Infinity LC ultra-high performance liquid chromatography (UHPLC) system.

Column temperature: 25° C.; flow rate: 0.5 mL/min; injection volume: 2 μL; mobile phase composition A: water+25 mM ammonium acetate+25 mM ammonia, B: acetonitrile; a gradient elution procedure is as follows: 0 min-0.5 min, 95% B; 0.5 min-7 min, B varied linearly from 95% to 65%; 7 min-8 min, B varied linearly from 65% to 40%; 8 min-9 min, B was maintained at 40%; 9 min-9.1 min, B varied linearly from 40% to 95%; 9.1 min-12 min, B was maintained at 95%; in the entire analysis process, the samples were placed in an auto sampler at 4° C.

To avoid an effect caused by fluctuation of a detection signal of the instrument, continuous analysis was performed on the samples in a random order. QC samples were inserted into a sample queue for monitoring and evaluating the stability of the system and the reliability of the experimental data.

### Conditions for Mass Spectrometry:

Primary and secondary spectrums of the samples were collected by using an AB Triple TOF 6600 mass spectrometer.

Conditions for an ESI source after the HILIC chromatographic separation are as follows: spray gas (Gas1): 60, auxiliary heating gas (Gas2): 60, curtain gas (CUR): 30, ion source temperature: 600° C., spray voltage (ISVF): ±5500 V (positive and negative modes); mass range of TOF MS scan: 60 Da-1000 Da; mass range of product scan: 25 Da-1000 Da; collection frequency of TOF MS scan: 0.20 s/spectra; collection frequency of product scan: 0.05 s/spectra.

The secondary mass spectrums were obtained in a data-dependent scanning mode (IDA), a high-sensitivity mode was used, declustering potential (DP): ±60 V (positive and negative modes), crash energy: 35±15 eV, the IDA is set as follows: the case where a relative molecular mass of an isotope ranged within 4 Da was ruled out, to-be-monitored candidate ions in each period: 10.

### (3) Metabolite Structure Determination

The collected raw data in a Wiff format were converted into the data in an .mzXML format by using ProteoWizard, and peak alignment, retention time correction and peak area extraction were performed by using XCMS software.

Metabolite structural identification and data preprocessing were performed on the data extracted by using the XCMS first, and then experimental data quality evaluation was performed.

### (4) Data Analysis

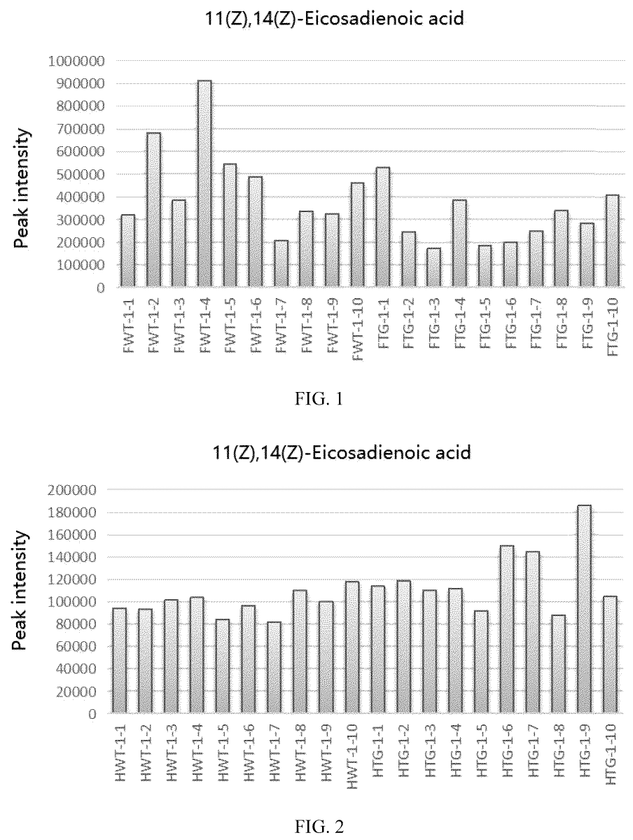

As can be seen from FIG. 1, significant differences are between the peak intensities of the mass spectrums of 11(Z), 14(Z)-eicosadienoic acid in the fecal metabolites of the AD model mice in the experimental group and the wild-type control mice (WT). Table 1 shows the comparison results of the fecal metabolites of the AD model mice and the wild-type control mice, where the VIP of 11(Z), 14(Z)-eicosadienoic acid is 3.28, and the p-value is 0.038. The comparative analysis results of the inter-group differences show that a content of 11(Z), 14(Z)-eicosadienoic acid in the AD model mice is significantly lower than that in the wild-type control mice.

TABLE 1

| Ratio of | | | | | |
|---|---|---|---|---|---|
| Experimental Group | | | Fold | | Signif- |
| to Control Group | Metabolite | VIP | Change | p-value | icance |
| APP/PS1 vs. WT | 11(Z),14(Z)- | 3.28 | 0.64 | 0.038 | * |
| | eicosadienoic | | | | |
| | acid | | | | |

Note:
“*” is P < 0.05.

### Example 2

This example provides a method for detecting an Alzheimer's disease biomarker in a hippocampus.

Compared with Example 1, samples of this example are hippocampi of brains of AD model mice and wild-type control mice.

### (1) Sample Processing

Hippocampus samples of brains were collected from 9-month-old AD model mice (APP/PS1) and wild-type control mice, and the samples were pretreated. After the collection, the samples were placed on dry ice for quick freezing and placed in a refrigerator at −80° C. for storage. After the samples were slowly thawed in an environment at 4° C., the to-be-detected samples were taken, a pre-cooled methanol/acetonitrile/aqueous solution (2:2:1, v/v) was added, vortexed to be mixed, sonicated for 30 min at a low temperature, left for 10 min at −20° C. and centrifuged at 14000 g for 20 min at 4° C., and supernatant was taken for vacuum drying. When mass spectrometry analysis was performed, 100 μL acetonitrile aqueous solution (acetonitrile:water=1:1, v/v) was added for reconstitution, vortexed and centrifuged at 14000 g for 15 min at 4° C., and supernatant was taken for the injection analysis.

### (2) Ultra-High Performance Liquid Chromatography-Mass Spectrometry Analysis

Separation was performed by using a HILIC column of an Agilent 1290 Infinity LC ultra-high performance liquid chromatography (UHPLC) system. Conditions for chromatography were the same as those in Example 1.

Primary and secondary spectrums of the samples were collected by using an AB Triple TOF 6600 mass spectrometer. Conditions for mass spectrometry were the same as those in Example 1.

### (3) Metabolite Structure Determination

The collected raw data in a Wiff format were converted into the data in an .mzXML format by using ProteoWizard, and peak alignment, retention time correction and peak area extraction were performed by using XCMS software.

Metabolite structural identification and data preprocessing were performed on the data extracted by using the XCMS first, and then experimental data quality evaluation was performed.

### (4) Data Analysis

As can be seen from FIG. 2, significant differences are between the peak intensities of the mass spectrums of 11(Z), 14(Z)-eicosadienoic acid in the hippocampi of the brains of the AD model mice in the experimental group and the wild-type control mice. Table 2 shows the comparison results of the hippocampal metabolites of the brains of the AD model mice and the wild-type control mice, where the VIP of 11(Z), 14(Z)-eicosadienoic acid is 1.08, and the p-value is 0.031. The comparative analysis results of the inter-group differences show that a content of 11(Z), 14(Z)-eicosadienoic acid in the hippocampi of the brains of the AD model mice is significantly higher than that of the wild-type control mice.

TABLE 2

| Ratio of | | | | | |
|---|---|---|---|---|---|
| Experimental Group | | | Fold | | Signif- |
| to Control Group | Metabolite | VIP | Change | p-value | icance |
| APP/PS1 vs. WT | 11(Z),14(Z)- | 1.08 | 1.24 | 0.031 | * |
| | eicosadienoic | | | | |
| | acid | | | | |

Note:
“*” is P < 0.05.

### Note: “*” is P<0.05,

As can be seen from Examples 1 and 2, the content of 11(Z), 14(Z)-eicosadienoic acid in the fecal metabolites of the AD model mice is significantly lower than that of the wild-type mice control group while the content of 11(Z), 14(Z)-eicosadienoic acid in the hippocampi of the AD model mice is significantly higher than that of the wild-type mice control group, suggesting that the change in the level of the fecal metabolites may reflect abnormalities of related metabolic pathways in the brains of the AD. Therefore, using 11(Z), 14(Z)-eicosadienoic acid as the Alzheimer's disease biomarker is of great significance for early clinical diagnosis of Alzheimer's disease.

### Example 3

This example provides an apparatus for predicting a risk of suffering from Alzheimer's disease.

The apparatus for predicting a risk of suffering from Alzheimer's disease includes:
a detection unit, which detected a content of 11(Z), 14(Z)-eicosadienoic acid in a to-be-detected sample through a liquid chromatography-mass spectrometry method;
a control unit, which was configured to perform second check, confirmation and experimental data quality evaluation on a result of qualitative and quantitative analysis of 11(Z), 14(Z)-eicosadienoic acid; and
an evaluation unit, which used the content of 11(Z), 14(Z)-eicosadienoic acid as an input variable, compared the input variable with a content of 11(Z), 14(Z)-eicosadienoic acid in a normal sample and provided a risk score after comprehensive calculation to assist in predicting the risk of suffering from Alzheimer's disease.

An evaluation criterion is as follows: a ratio of the content of 11(Z), 14(Z)-eicosadienoic acid in the to-be-detected sample to the content of 11(Z), 14(Z)-eicosadienoic acid in the normal sample was less than 1, then it was determined that the to-be-detected sample suffered from or was at risk of suffering from Alzheimer's disease.

The content of 11(Z), 14(Z)-eicosadienoic acid in the stool sample of the to-be-detected organism was detected, and it was determined that whether the to-be-detected sample suffered from or was at risk of suffering from Alzheimer's disease according to the ratio of the content of 11(Z), 14(Z)-eicosadienoic acid in the to-be-detected sample to the content of 11(Z), 14(Z)-eicosadienoic acid in the normal sample.

In the present application, the Alzheimer's disease biomarker has a relatively low detection amount in the feces of the mice suffering from Alzheimer's disease. Similarly, when the detection samples are extended to a patient population suffering from Alzheimer's disease, the same detection result is also obtained. This detection method, which uses 11(Z), 14(Z)-eicosadienoic acid as the biomarker, is characterized by high detection accuracy, convenience, quickness, safety and non-invasiveness, which is of great clinical guiding significance for auxiliary diagnosis of a relevant index of Alzheimer's disease.

The applicant states that the above are the specific examples of the present application and not intended to limit the protection scope of the present application. Those skilled in the art should understand that any changes or substitutions easily conceivable by those skilled in the art within the technical scope disclosed in the present application fall within the protection scope and the disclosed scope of the present application.

What is claimed is:

1. An Alzheimer's disease biomarker, which is 11(Z), 14(Z)-eicosadienoic acid.

2. An early diagnosis model for Alzheimer's disease, wherein an input variable of the early diagnosis model for Alzheimer's disease comprises a peak intensity value of a mass spectrum of the Alzheimer's disease biomarker 11(Z), 14(Z)-eicosadienoic acid according to claim 1.

3. The early diagnosis model for Alzheimer's disease according to claim 2, wherein an output variable of the early diagnosis model for Alzheimer's disease comprises a ratio of a peak intensity value of a mass spectrum of 11(Z), 14(Z)-eicosadienoic acid in a stool sample of a to-be-detected individual to a peak intensity value of a mass spectrum of 11(Z), 14(Z)-eicosadienoic acid in a stool sample of a normal individual, wherein in a case where the ratio is less than 1, the to-be-detected individual is judged to suffer from or be at risk of suffering from Alzheimer's disease.

4. A screening method for the Alzheimer's disease biomarker according to claim 1, comprising: collecting a sample, detecting the sample, and performing structural identification and data analysis on a metabolite in the sample.

5. The screening method according to claim 4, wherein in the step of collecting the sample, the sample comprises feces; and
in the step of detecting the sample, the detection is performed through a liquid chromatography-mass spectrometry method.

6. The screening method according to claim 4, wherein in the step of performing the structural identification on the metabolite in the sample, the structural identification is performed on the metabolite in the sample by matching with a retention time, a molecular mass, a secondary fragmentation spectrum and crash energy of a metabolite in a database, and an obtained identification result is checked and confirmed; and
optionally, in the step of performing the data analysis on the metabolite in the sample, the data analysis is performed on the metabolite in the sample through any one or a combination of at least two of univariate statistical analysis, multidimensional statistical analysis, differential metabolite screening, differential metabolite correlation analysis or KEGG pathway analysis.

7. The screening method according to claim 6, wherein the multidimensional statistical analysis comprises: constructing an orthogonal partial least squares-discriminant analysis model, and screening out a metabolite with VIP>1 from a detection result as the Alzheimer's disease biomarker.

8. (canceled)

9. An apparatus for predicting a risk of suffering from Alzheimer's disease, comprising:
a detection unit, which is configured to perform qualitative and quantitative analysis on the Alzheimer's disease biomarker according to claim 1 in a metabolite sample;
a control unit, which is configured to perform second check, confirmation and experimental data quality evaluation on a result of the qualitative and quantitative analysis of the Alzheimer's disease biomarker; and
an evaluation unit, which uses a quantitative result of the Alzheimer's disease biomarker as an input variable and provides a risk score after comprehensive calculation to predict the risk of suffering from Alzheimer's disease;
wherein the detection unit performs the qualitative and quantitative analysis on the Alzheimer's disease biomarker through a liquid chromatography-mass spectrometry method.

10. A diagnostic kit for Alzheimer's disease, comprising a reagent for measuring a content level of the Alzheimer's disease biomarker according to claim 1.

11. (canceled)

12. A method for treating or diagnosing Alzheimer's disease, comprising administering an effective amount of the Alzheimer's disease biomarker according to claim 1 to subject in need thereof.
